(19) United States
(12) Patent Application Publication
(10) Pub. No.: US 2022/0013198 A1
(43) Pub. Date: Jan. 13, 2022

(54) ENHANCED MONITORING OF CLINICAL TRIALS

(71) Applicant: International Business Machines Corporation, Armonk, NY (US)

(72) Inventor: William Scott Spangler, San Martin, CA (US)

(21) Appl. No.: 16/924,371

(22) Filed: Jul. 9, 2020

(51) Int. Cl. G16H 10/20; G06F 16/2455; G16H 50/70; G16H 70/60; G16H 40/20; G16H 50/20; G16H 70/20; G16H 15/00

(52) U.S. Cl. CPC G16H 10/20; G06F 16/24556; G16H 50/70; G16H 15/00; G16H 40/20; G16H 50/20; G16H 70/20; G16H 70/60

(57) ABSTRACT

A set of clinical trial information associated with a clinical trial process is monitored. In response to the monitoring, a clinical trial status is generated. The clinical trial status and at least a subset of the set of clinical trial information is aggregated with a plurality of historical clinical trial statuses and information. From the aggregation, a queryable knowledge base describing each clinical trial status and information within the aggregation is generated. In response to the generation of the queryable knowledge base, a user is notified of at least one of the aggregation of clinical trial statuses and information.

### BACKGROUND

The present disclosure relates generally to the field of clinical trials, and more particularly to preventing information loss among clinical trials.

Scientific publications tend to be biased toward positive outcomes. The nature of scientific research discourages repetition of failed studies or negative results. Clinical trials proceed in phases, and the mere fact that a particular trial does not advance to the next phase does not automatically indicate failure.

Neural networks and machine learning are becoming more and more prevalent in several aspects of computer science. Machine learning models may be used for a wide variety of applications, such as “reading” handwritten documents, object and/or facial recognition techniques, generating and calculating algorithms, etc.

### SUMMARY

Embodiments of the present disclosure include a method, computer program product, and system for preventing information loss among clinical trials.

A set of clinical trial information associated with a clinical trial process is monitored. In response to the monitoring, a clinical trial status is generated. The clinical trial status and at least a subset of the set of clinical trial information is aggregated with a plurality of historical clinical trial statuses and information. From the aggregation, a queryable knowledge base describing each clinical trial status and information within the aggregation is generated. In response to the generation of the queryable knowledge base, a user is notified of at least one of the aggregation of clinical trial statuses and information.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 illustrates a high-level diagram of an example computing environment for preventing information loss among clinical trials, in accordance with embodiments of the present disclosure.

FIG. 2 illustrates a flowchart of a method for preventing information loss among clinical trials, in accordance with embodiments of the present disclosure.

FIG. 3 illustrates a flowchart of a method for querying a knowledge base for preventing information loss among clinical trials, in accordance with embodiments of the present disclosure.

FIG. 4 illustrates an example neural network that may be used to extract features and/or generate cross-references among features, in accordance with embodiments of the present disclosure.

FIG. 5 depicts a cloud computing environment according to an embodiment of the present disclosure.

FIG. 6 depicts abstraction model layers according to an embodiment of the present disclosure.

FIG. 7 depicts a high-level block diagram of an example computer system that may be used in implementing embodiments of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of clinical trials, and more particularly to preventing information loss among clinical trials. While the present disclosure is not necessarily limited to such applications, various aspects of the disclosure may be appreciated through a discussion of various examples using this context.

Scientific publications tend to be biased towards positive outcomes. Scientists, like most people, are less interested in talking about failures than successes. Journals, conferences, and other publishers focus primarily on novel discoveries, and traditionally discourage reporting negative results, or reject such publications when they are submitted. This presents a problem when individual scientists try to understand the current state of knowledge about a given hypothesis. Since many past experimental results may be unreproducible or simply wrong, it is usually unwise to accept the results of individual studies at face value. Embodiments of the present disclosure contemplate “reading between the lines” of a set of all published abstracts and clinical trials, over time, to detect research dead ends and make them explicit for the scientist or other user. By better documenting and representing scientific failures, and the state of clinical trials and other studies in general, users may avoid repeating the same mistakes, or identify unpursued research opportunities.

Conventional approaches to monitoring the state of clinical trials (e.g., manually curated databases) have been used to try to summarize the results of past treatment protocols and research. This is high in time and labor costs and not very comprehensive or consistent in defining “failure” and other parameters.

Embodiments of the present disclosure contemplate the automated generation of a queryable knowledge base to prevent information loss related to progression of research and clinical trials. In some embodiments, clinical trials are classified by one or more features (e.g., disease ontology, gene ontology, chemical structure, biologic, course of treatment, therapy, indication, etc.). The clinical trials (e.g., for a given treatment-indication pair) may be tracked over time, and those with express terminations may be designated as “failures.” The clinical trials that progress through the phases (traditionally, clinical trials progress from “phase one,” to “phase two,” to “phase three,” and then to “approved,” but this disclosure should not be read to limit itself to a three/four phase process) may be designated as “successes.” Some clinical trials may progress, but the next phase in the process may be for a different indication. In such instances, the clinical trial could be classified as a “continuation.” Lastly, some clinical trials may pass a particular phase, but the next phase may never be initiated. In such an event, such a trial may be designated “not pursued,” after a particular time period passes (e.g., two years).

The successes, failures, continuations and not pursueds may be aggregated into a queryable knowledge base, along with any additional information that may be useful (e.g., associated biomarker(s), method of action, pharmacokinetics, etc.).

In some embodiments, a user may query the knowledge base, according to any of the clinical trial classifications, to return a list of clinical trial information that may be pertinent to the query. In some embodiments, the knowledge base entries may be cross-referenced to each other. For example, a single chemical compound may be a feature of two different clinical trials with two different indications. When one of the two indications is queried, the second indication may be addended to the search results via the cross-referencing. In some embodiments, a neural network may be employed to extract features and/or to generate cross-referencing links within the knowledge base.

As an example, by assigning each clinical trial a normalized treatment and indication, the information on treatment/gene, gene/disease, treatment/disease relationships extracted from literature can be used to leverage past clinical trials in order to evaluate the overall effectiveness of treatment strategies. For example, given a new therapeutic/treatment which targets a particular gene for a particular disease, all past clinical trials where a treatment targeted that gene for that disease and provide a success/(success+failure) score for that approach can be looked up. This can be broadened out further to look for other conditions with a similar genetic profile to the disease of interest and find additional trials that are relevant.

An example of java code that could be used to implement embodiments of the present disclosure may include, in part:

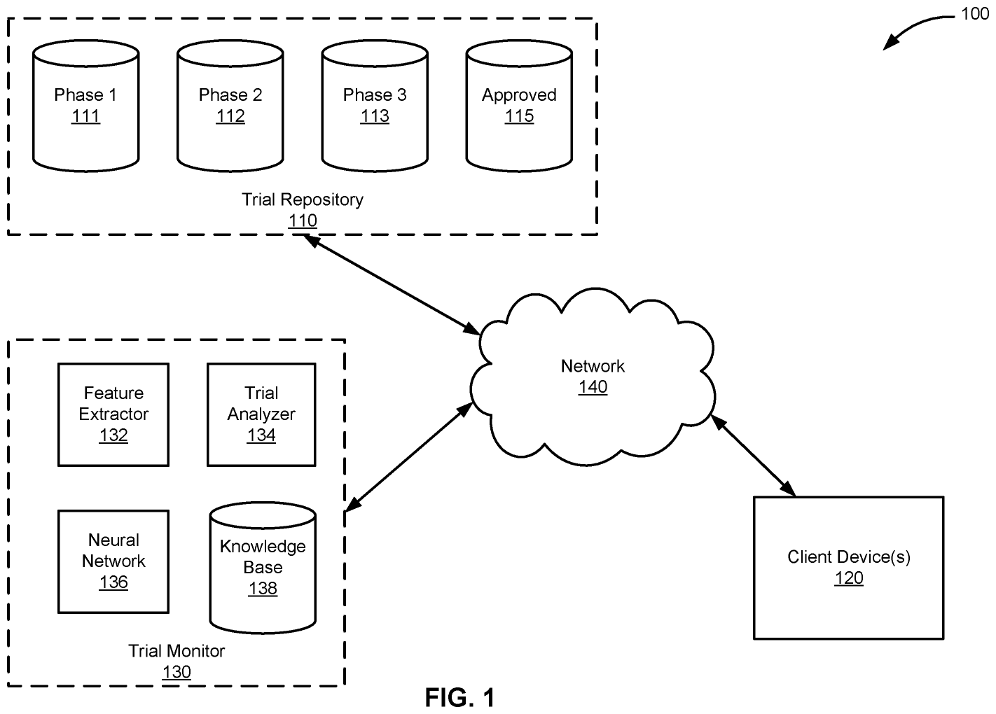

Referring now to FIG. 1, illustrated is a high-level diagram of an example computing environment 100 for preventing information loss among clinical trials, in accordance with embodiments of the present disclosure. Example computing environment 100 may be implemented as one or more physical devices (e.g., desktop computers, smart phones, tablets, etc.) communicatively coupled to each other (or even potentially a single standalone system), or it may be implemented in some degree using a cloud computing environment where one or more components of the environment are virtualized and run on a set of remote devices operating to sustain the virtualized components via one or more hypervisors. In any of these embodiments, data may be transferred using a physical or wireless network (e.g., network 140) of any suitable configuration and using any suitable communications protocol(s). In some embodiments, encryption may be employed to secure the communications and maintain privacy.

In some embodiments, example computing environment 100 may further include one or more client device(s) 120, trial repository 110, and trial monitor 130. In some embodiments, client device 120 may include, for example, a smartphone, tablet, desktop computer, etc. communicably coupled to trial monitor 130 and/or trial repository 110. In yet other embodiments, client device 120 may include a microphone or other input peripheral, and the client device 120 may be incorporated into a standalone trial monitoring device (e.g., a device including client device 120, trial monitor 130, and trial repository 110).

Trial repository 110 may include, for example, a database or other collection of clinical trials and their associated information (e.g., the database at clinicaltrials.gov). While trial repository 110 is shown with clinical trial information already parsed into phase 1 111, phase 2 112, phase 3 113, and approved 115, those having skill in the art will appreciate that the clinical trial information within trial repository 110 may not, and need not, be pre-sorted or parsed into such classifications. Some embodiments of the present disclosure are capable of performing such sorting on their own, and the depiction of trial repository 110 in this manner is for ease of description and should not be construed as limiting in any way.

In some embodiments, phase 1 111 represents the clinical trial information associated with clinical trials that have participated in phase 1. Phase 1 clinical trials typically include testing a new t (e.g., chemical compound, biologic, etc.) in healthy volunteers. The primary purpose of a phase 1 clinical trial is to evaluate the safety of the treatment, as well as determine blood level concentrations during administration, how the treatment works in the body, and any side effects from increased dosage(s).

In some embodiments, phase 2 112 represents the clinical trial information associated with clinical trials that have participated in phase 2. Phase 2 clinical trials typically include administration of the treatment to a larger group of volunteers who have the disease or condition for which the treatment is being developed. Phase 2 may include further assessment of effectivity and safety (e.g., treatment effectivity), as well as a determination of optimal dose(s).

In some embodiments, phase 3 113 represents the clinical trial information associated with clinical trials that have participated in phase 3. Phase 3 clinical trials typically include an even larger group of volunteers, also from patient populations for which the treatment is being developed. Volunteers may be assigned to receive the treatment or a placebo in order to demonstrate whether the treatment has a statistically significant benefit and to provide enhanced safety data (e.g., for product labeling and other purposes).

In some embodiments, approved 115 represents the clinical trial information associated with clinical trials that have approval from a regulatory body. Approved 115 may include information generated from every other phase of the process, as well as any other information (e.g., information generated during a New Drug Application (NDA) process) required by the regulatory body (e.g., the Food and Drug Administration (FDA)).

In some embodiments, trial monitor 130 may include, for example, a feature extractor 132, trial analyzer 134, neural network 136, and knowledge base 138. Feature extractor 132 may allow the trial monitor 130 to extract features of clinical trials from any of the phases from trial repository 110. Clinical trial features may include, for example, disease ontology, gene ontology, chemical structure, biologic, course of treatment, therapy, indication, etc. Feature extractor 132 may employ, for example, natural language processing (NLP) techniques to generate machine readable data from clinical trial information. In some embodiments, neural network 136 may be employed to aid feature extractor 132 in the identification of features. This may be particularly helpful when extracting features associated with graphs or other visual elements within clinical trial information (e.g., pharmacokinetics, optimal dose graphs, etc.).

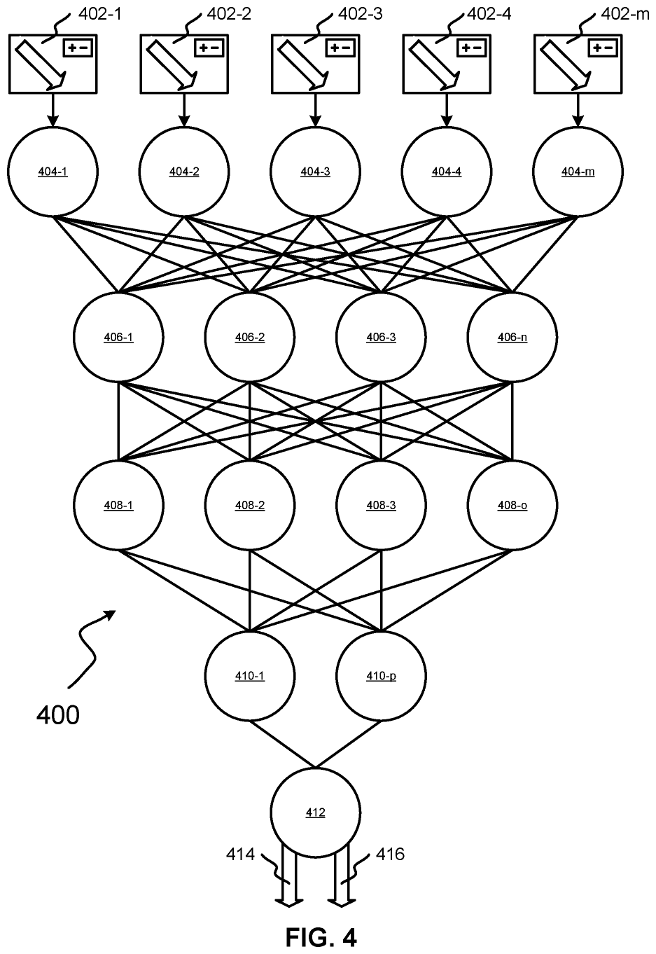

Trial analyzer 134 may ingest the features and other clinical trial information to determine cross-referencing information among the various trials, as well as determine the classification of the clinical trial described (e.g., successes, failures, continuations and not pursueds), and further aggregate the information into a queryable knowledge base (e.g., knowledge base 138), along with any additional information that may be useful (e.g., associated biomarker(s), method of action, pharmacokinetics, etc.) In some embodiments, neural network 136 may assist trial analyzer 134 in the classification of clinical trials and/or in the generation of cross-reference links. Further detail is provided with regard to neural networks (e.g., neural network 136) in the description of FIG. 4.

Knowledge base 138 may be a database or other storage scheme (e.g., relational database, triplestore, text index, etc.) for storing clinical trial information and associated additional information (e.g., cross-references and/or metadata). Knowledge base 138 may include clinical trial information sorted by classification (e.g., success, failure, continuation, not pursued), feature (e.g., disease ontology, gene ontology, chemical structure, biologic, course of treatment, therapy, indication, etc.), or any other aspect of the described information (e.g., associated biomarker(s), method of action, pharmacokinetics, etc.).

In some embodiments, the information stored in knowledge base 138 may be used to generate, for example, an interactive knowledge graph which may be queried according to any pertinent information field/feature/class (e.g., chemical structure, disease ontology, gene ontology, indication, biomarker(s), treatment, etc.), and filtered using a second information field/feature/class. In some embodiments, other associated clinical studies (e.g., clinical information that has been cross-referenced within the knowledge base 138) may be returned in response to the query, along with the query's actual results (e.g., in the form of footnotes, a side-window or popup, etc.). In some embodiments, a library of chemical structures (not shown) may be used to determine the various names for similar or identical compounds. For example, a single compound may have a brand name, a common chemical structure name, an IUPAC (International Union of Pure and Applied Chemistry) name, etc.

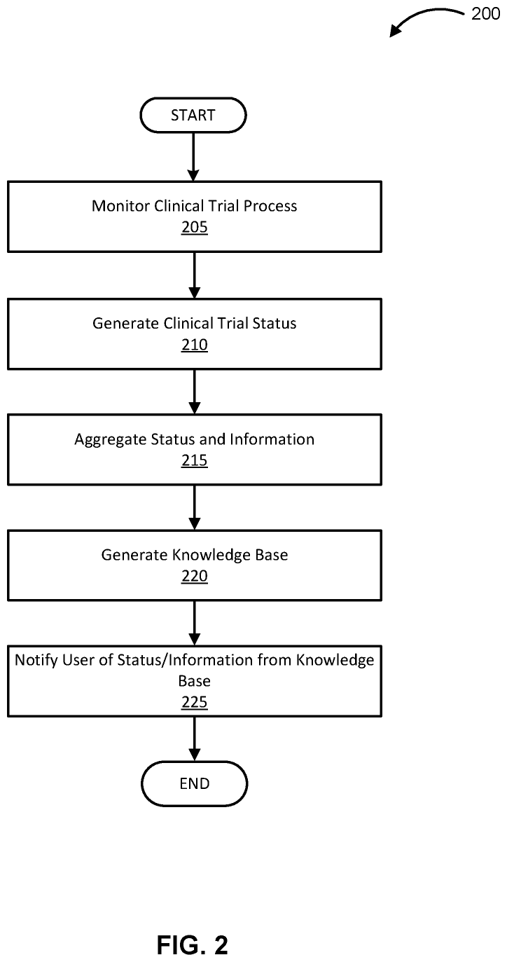

Referring now to FIG. 2, illustrated is a flowchart of a method 200 for preventing information loss among clinical trials, in accordance with embodiments of the present disclosure. Method 200 may begin at 205, where a clinical trial process is monitored. In some embodiments, this may include monitoring a process among the various phases for a particular treatment-indication pair. This may include monitoring the overall process (phase 1->phase 2->phase 3->approved), or it may include a subset of the overall process.

At 210, a clinical trial status is generated. In some embodiments, the clinical trial status me be referred to as a completion assessment. In some embodiments, the clinical trial status may correspond to the clinical trial classification, as described herein (e.g., success, failure, continuation, not pursued, etc.).

At 215, the clinical trial status and at least a subset of the clinical trial information is aggregated, according to embodiments. The clinical trial information may include, for example, extracted features and additional information, as described herein, or any other information associated with a pertinent clinical trial. In some embodiments, the aggregation may include historical clinical trial information from, for example, one or more regulatory databases (e.g., clinicaltrials.gov), as well as proprietary and/or private databases. In some embodiments, the aggregation may further include research studies and other resources not included in the clinical trial process (e.g., public health surveys, clinical practice research, clinical laboratory research, establishment of reference ranges among testing sample types, etc.).

At 220, a queryable knowledge base containing the aggregated information, as well as any cross-referencing information (e.g., metadata annotations or other relationships among various clinical trials and their information) may be generated. As described herein, the knowledge base may include a triplestore, relational database, text index, etc.

At 225, a user is notified of at least one clinical trial status and information from the aggregation. In some embodiments, the notification may be in response to a user-generated query. In yet other embodiments, the notification may be automatic (e.g., based on an analysis of a user profile) and/or periodic (e.g., once per month). In some embodiments, a notification for a plurality of users may be sent concurrently, using parallel techniques (e.g., Single Data Multiple Instruction (SIMD)) for conservation of computing resources.

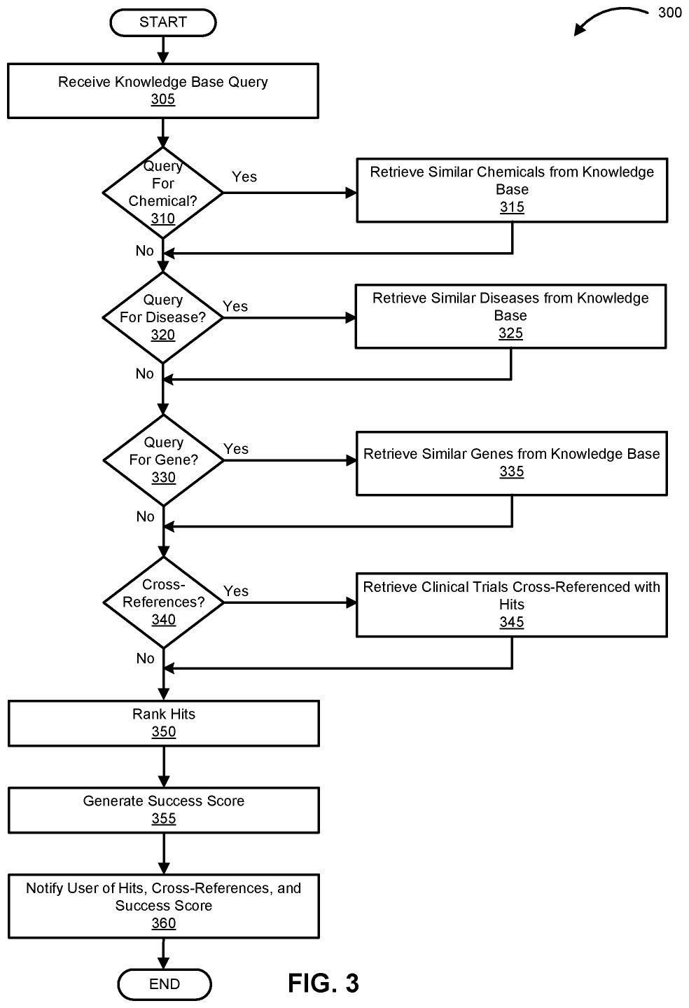

Referring now to FIG. 3, illustrated is a flowchart of a method 300 for querying a knowledge base for preventing information loss among clinical trials, in accordance with embodiments of the present disclosure. Method 300 may begin at 305, where a query for the knowledge base is received. In some embodiments, a query may be received via interaction with a specialized graphical user interface (GUI) (e.g., a knowledge graph) depicting the information stored in the knowledge base.

At 310, it is determined whether the query includes a chemical structure/name. If yes, the method proceeds to 315, where the chemical structure/name is retrieved from the knowledge base. In some embodiments, this retrieval may include knowledge base entries with similar/identical chemical structures/names (e.g., brand names, IUPAC names, enantiomers, salts (e.g., HBr, HCl, etc.), etc.). In some embodiments, chemical structure/name variants may be included in the cross-reference retrieval discussed at 345.

If, however, the query does not include a chemical structure/name, the method may proceed to 320, where it is determined whether the query includes a disease. If yes, then the method may proceed to 325, where knowledge base entries for the disease are retrieved. In some embodiments, similar/identical diseases with a different name may also be retrieved (e.g., studies/trials from different locations may refer to the same condition/disease by a different name and/or spelling). In some embodiments, disease name variants may be included in the cross-reference retrieval discussed at 345.

If, however, the query does not include a disease name, the method may proceed to 330, where it is determined whether the query includes a gene. If yes, then the method may proceed to 335, where knowledge base entries for the gene are retrieved. In some embodiments, similar/identical genes with a different name may also be retrieved (e.g., multiple genes may be concurrently discovered/named by different parties, two different genes may regulate a similar cellular function, gene mutations of the queried gene, etc.). In some embodiments, gene variants may be included in the cross-reference retrieval discussed at 345.

If, however, the query does not include a gene, the method may proceed to 340, where it is determined whether the retrieved knowledge base entry/entries include cross-references to any other entries. If yes, then the method may proceed to 345, where knowledge base entries associated with those cross-references are retrieved. In some embodiments, this may include a single degree of relationship (e.g., only entries directly cross-referenced to a retrieved entry), or it may include multiple degrees of relationship (e.g., entries indirectly cross-referenced to the retrieved entries via a third/fourth/fifth/etc. entry).

It should be understood that while method 300 is limited to a depiction of queries for chemical/disease/gene/cross-reference, it should be understood that any clinical trial status/classification, clinical trial feature, or additional information may be used to query the knowledge base, and therefore the examples given here should not be construed as limiting the disclosure in any way.

At 350, the hits (e.g., retrieved entries) may be ranked. This may include a determination of which retrieved entries are most relevant to the original query. In embodiments, this may include sorting the list of retrieved entries and/or cross-references.

At 355, a success score is generated, as discussed herein (e.g., success/(success+failure) for all historical clinical trial entries within the knowledge base that are retrieved by the query). In some embodiments, a neural network may be employed to generate a success score, and additional factors (e.g., date of trial, number of volunteers/participants in the trial, whether a confirmation trial or study has validated the results, etc.) may be used to generate the success score. In some embodiments, some factors may be weighted.

At 360, the user from whom the query was received is notified of the hits, cross-references, and success score for the query. This may be accomplished in any suitable manner, for example, a popup, text message, e-mail, via an interactive knowledge graph GUI, etc.

FIG. 4 depicts an example neural network 400 that may be used to perform several of the functions disclosed herein (e.g., classification of clinical trials, extraction of features, generation of cross-references, generation of success scores, etc.), in accordance with embodiments of the present disclosure. The example neural network 400 may further be communicably linked to one or more user devices, one or more trial monitors, one or more trial repositories, and/or one or more other neural networks. In embodiments, parallel techniques (e.g., Single Instruction Multiple Data (SIMD) techniques) may be employed to concurrently generate vectors of the same type.

In embodiments, neural network 400 may be a classifier-type neural network. Neural network 400 may be part of a larger neural network (e.g., may be a sub-unit of a larger neural network). For example, neural network 400 may be nested within a single, larger neural network, connected to several other neural networks, or connected to several other neural networks as part of an overall aggregate neural network.

Inputs 402-1 through 402-m represent the inputs to neural network 400. In this embodiment, 402-1 through 402-m do not represent different inputs. Rather, 402-1 through 402-m represent the same input that is sent to each first-layer neuron (neurons 404-1 through 404-m) in neural network 400. In some embodiments, the number of inputs 402-1 through 402-m (i.e., the number represented by m) may equal (and thus be determined by) the number of first-layer neurons in the network. In other embodiments, neural network 400 may incorporate 1 or more bias neurons in the first layer, in which case the number of inputs 402-1 through 402-m may equal the number of first-layer neurons in the network minus the number of first-layer bias neurons. In some embodiments, a single input (e.g., input 402-1) may be input into the neural network. In such an embodiment, the first layer of the neural network may comprise a single neuron, which may propagate the input to the second layer of neurons.

Inputs 402-1 through 402-m may comprise one or more samples of classifiable data. For example, inputs 402-1 through 402-m may comprise 10 samples of classifiable data. In other embodiments, not all samples of classifiable data may be input into neural network 400.

Neural network 400 may comprise 5 layers of neurons (referred to as layers 404, 406, 408, 410, and 412, respectively corresponding to illustrated nodes 404-1 to 404-m, nodes 406-1 to 406-n, nodes 408-1 to 408-o, nodes 410-1 to 410-p, and node 412). In some embodiments, neural network 400 may have more than 5 layers or fewer than 5 layers. These 5 layers may each be comprised of the same number of neurons as any other layer, more neurons than any other layer, fewer neurons than any other layer, or more neurons than some layers and fewer neurons than other layers. In this embodiment, layer 412 is treated as the output layer. Layer 412 outputs a probability that a target event will occur and contains only one neuron (neuron 412). In other embodiments, layer 412 may contain more than 1 neuron. In this illustration no bias neurons are shown in neural network 400. However, in some embodiments each layer in neural network 400 may contain one or more bias neurons.

Layers 404-412 may each comprise an activation function. The activation function utilized may be, for example, a rectified linear unit (ReLU) function, a SoftPlus function, a Soft step function, or others. Each layer may use the same activation function, but may also transform the input or output of the layer independently of or dependent upon the activation function. For example, layer 404 may be a “dropout” layer, which may process the input of the previous layer (here, the inputs) with some neurons removed from processing. This may help to average the data and can prevent overspecialization of a neural network to one set of data or several sets of similar data. Dropout layers may also help to prepare the data for “dense” layers. Layer 406, for example, may be a dense layer. In this example, the dense layer may process and reduce the dimensions of the feature vector (e.g., the vector portion of inputs 402-1 through 402-m) to eliminate data that is not contributing to the prediction. As a further example, layer 408 may be a “batch normalization” layer. Batch normalization may be used to normalize the outputs of the batch-normalization layer to accelerate learning in the neural network. Layer 410 may be any of a dropout, hidden, or batch-normalization layer. Note that these layers are examples. In other embodiments, any of layers 404 through 410 may be any of dropout, hidden, or batch-normalization layers. This is also true in embodiments with more layers than are illustrated here, or fewer layers.

Layer 412 is the output layer. In this embodiment, neuron 412 produces outputs 414 and 416. Outputs 414 and 416 represent complementary probabilities that a target event will or will not occur. For example, output 414 may represent the probability that a target event will occur, and output 416 may represent the probability that a target event will not occur. In some embodiments, outputs 414 and 416 may each be between 0.0 and 1.0, and may add up to 1.0. In such embodiments, a probability of 1.0 may represent a projected absolute certainty (e.g., if output 414 were 1.0, the projected chance that the target event would occur would be 100%, whereas if output 416 were 1.0, the projected chance that the target event would not occur would be 100%).

In embodiments, FIG. 4 illustrates an example probability-generator neural network with one pattern-recognizer pathway (e.g., a pathway of neurons that processes one set of inputs and analyzes those inputs based on recognized patterns, and produces one set of outputs). However, some embodiments may incorporate a probability-generator neural network that may comprise multiple pattern-recognizer pathways and multiple sets of inputs. In some of these embodiments, the multiple pattern-recognizer pathways may be separate throughout the first several layers of neurons, but may merge with another pattern-recognizer pathway after several layers. In such embodiments, the multiple inputs may merge as well. This merger may increase the ability to identify correlations in the patterns identified among different inputs, as well as eliminate data that does not appear to be relevant.

In embodiments, neural network 400 may be trained/adjusted (e.g., biases and weights among nodes may be calibrated) by inputting feedback and/or input from a to correct/force the neural network to arrive at an expected output. In some embodiments, the feedback may be forced selectively to particular nodes and/or sub-units of the neural network. In some embodiments, the impact of the feedback on the weights and biases may lessen over time, in order to correct for inconsistencies among user(s) and/or datasets. In embodiments, the degradation of the impact may be implemented using a half-life (e.g., the impact degrades by 50% for every time interval of X that has passed) or similar model (e.g., a quarter-life, three-quarter-life, etc.).

It is to be understood that although this disclosure includes a detailed description on cloud computing, implementation of the teachings recited herein are not limited to a cloud computing environment. Rather, some embodiments of the present invention are capable of being implemented in conjunction with any other type of computing environment now known or later developed.

Cloud computing is a model of service deliver for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources, but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure, but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities, but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

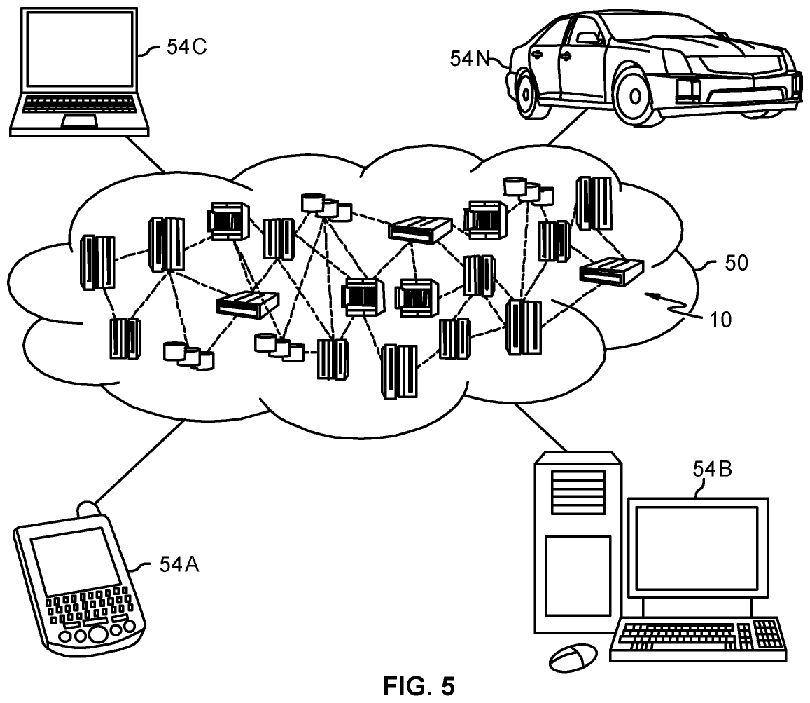

Referring now to FIG. 5, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

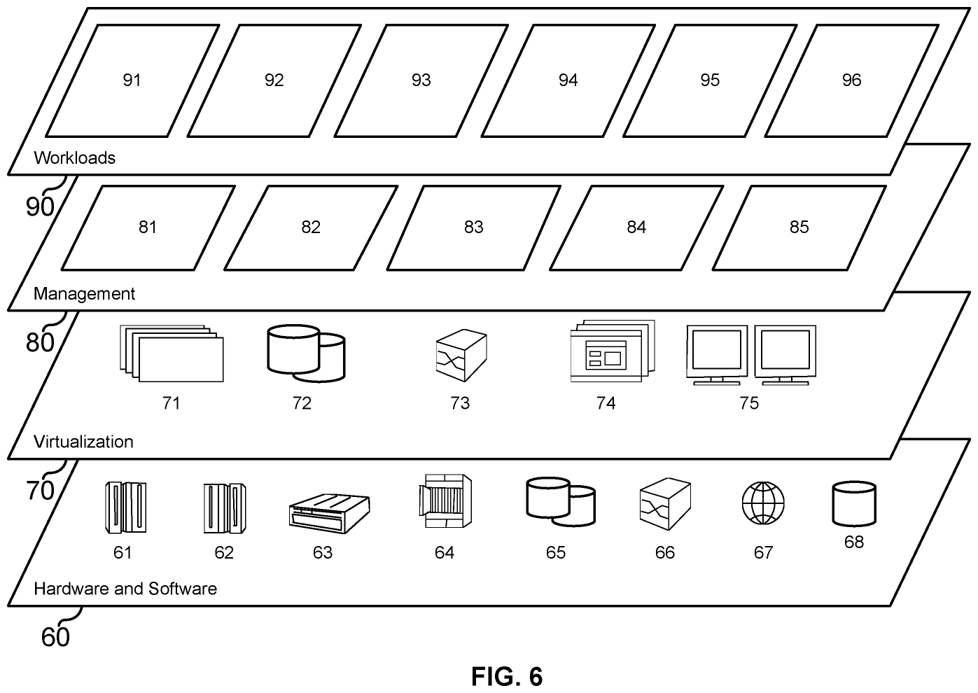

Referring now to FIG. 6, a set of functional abstraction layers provided by cloud computing environment 50 (FIG. 5) is shown. It should be understood in advance that the components, layers, and functions shown in FIG. 6 are intended to be illustrative only and some embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and enhanced monitoring of clinical trials 96.

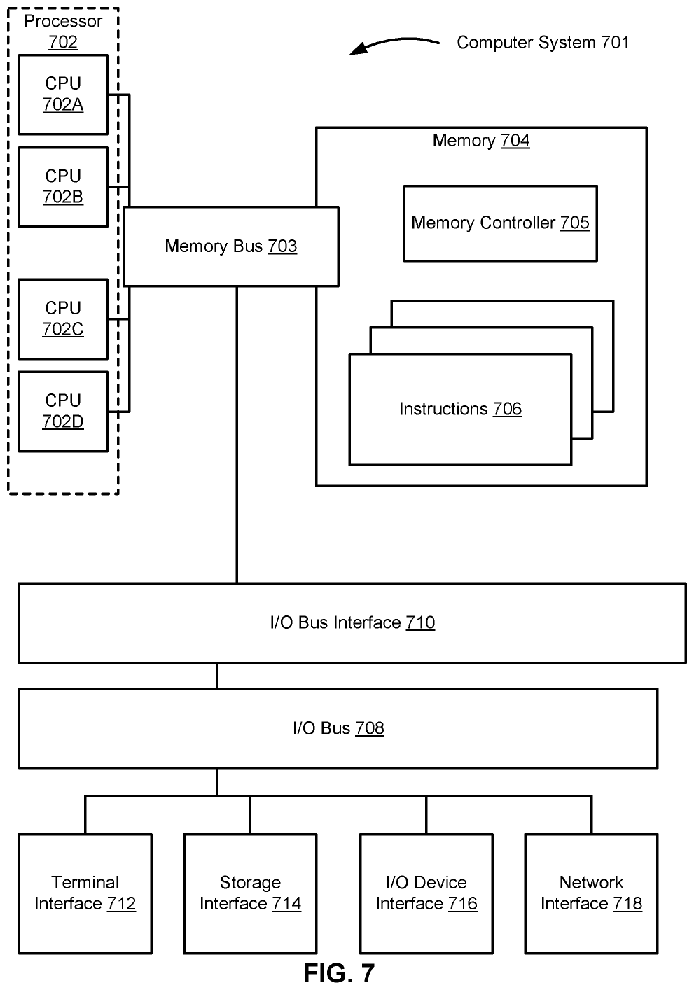

Referring now to FIG. 7, shown is a high-level block diagram of an example computer system 701 that may be configured to perform various aspects of the present disclosure, including, for example, methods 200/300, described in FIGS. 2-3. The example computer system 701 may be used in implementing one or more of the methods or modules, and any related functions or operations, described herein (e.g., using one or more processor circuits or computer processors of the computer), in accordance with embodiments of the present disclosure. In some embodiments, the illustrative components of the computer system 701 comprise one or more CPUs 702, a memory subsystem 704, a terminal interface 712, a storage interface 714, an I/O (Input/Output) device interface 716, and a network interface 718, all of which may be communicatively coupled, directly or indirectly, for inter-component communication via a memory bus 703, an I/O bus 708, and an I/O bus interface unit 710.

The computer system 701 may contain one or more general-purpose programmable central processing units (CPUs) 702A, 702B, 702C, and 702D, herein generically referred to as the CPU 702. In some embodiments, the computer system 701 may contain multiple processors typical of a relatively large system; however, in other embodiments the computer system 701 may alternatively be a single CPU system. Each CPU 702 may execute instructions stored in the memory subsystem 704 and may comprise one or more levels of on-board cache. Memory subsystem 704 may include instructions 706 which, when executed by processor 702, cause processor 702 to perform some or all of the functionality described above with respect to FIGS. 1-3.

In some embodiments, the memory subsystem 704 may comprise a random-access semiconductor memory, storage device, or storage medium (either volatile or non-volatile) for storing data and programs. In some embodiments, the memory subsystem 704 may represent the entire virtual memory of the computer system 701, and may also include the virtual memory of other computer systems coupled to the computer system 701 or connected via a network. The memory subsystem 704 may be conceptually a single monolithic entity, but, in some embodiments, the memory subsystem 704 may be a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory may be further distributed and associated with different CPUs or sets of CPUs, as is known in any of various so-called non-uniform memory access (NUMA) computer architectures. In some embodiments, the main memory or memory subsystem 704 may contain elements for control and flow of memory used by the CPU 702. This may include a memory controller 705.

Although the memory bus 703 is shown in FIG. 7 as a single bus structure providing a direct communication path among the CPUs 702, the memory subsystem 704, and the I/O bus interface 710, the memory bus 703 may, in some embodiments, comprise multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 710 and the I/O bus 708 are shown as single respective units, the computer system 701 may, in some embodiments, contain multiple I/O bus interface units 710, multiple I/O buses 708, or both. Further, while multiple I/O interface units are shown, which separate the I/O bus 708 from various communications paths running to the various I/O devices, in other embodiments some or all of the I/O devices may be connected directly to one or more system I/O buses.

In some embodiments, the computer system 701 may be a multi-user mainframe computer system, a single-user system, or a server computer or similar device that has little or no direct user interface, but receives requests from other computer systems (clients). Further, in some embodiments, the computer system 701 may be implemented as a desktop computer, portable computer, laptop or notebook computer, tablet computer, pocket computer, telephone, smart phone, mobile device, or any other appropriate type of electronic device.

It is noted that FIG. 7 is intended to depict the representative example components of an exemplary computer system 701. In some embodiments, however, individual components may have greater or lesser complexity than as represented in FIG. 7, components other than or in addition to those shown in FIG. 7 may be present, and the number, type, and configuration of such components may vary.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for characterizing the outcome of clinical trials, the method comprising:
monitoring a clinical trial process associated with a set of clinical trial information;
generating, in response to the monitoring, a clinical trial status;
aggregating the clinical trial status and at least a subset of the set of clinical trial information with a plurality of historical clinical trial statuses and information;
generating, from the aggregation, a queryable knowledge base describing each of the aggregation of clinical trial statuses and information; and
notifying, in response to the generation of the queryable knowledge base, a user of at least one of the aggregation of clinical trial statuses and information.

2. The method of claim 1, wherein the clinical trial information further includes at least one feature selected from the group consisting of disease ontology, gene ontology, chemical structure, biologic, course of treatment, therapy, and indication.

3. The method of claim 1, wherein the clinical trial status includes an evaluation of the clinical trial process into a completion assessment, wherein the completion assessment is selected from the group consisting of terminated, not pursued, completed, and continued.

4. The method of claim 2, wherein the clinical trial information for each aggregation includes a treatment-indication pair, and wherein notifying the user further comprises:
receiving, from the user, a query including a first treatment-indication pair;
identifying, according to the query, a subset of the aggregation associated with the first treatment-indication pair; and
notifying the user of the identified subset.

5. The method of claim 4, wherein the identifying further comprises generating, from the identified subset, a treatment effectivity for each aggregation within the subset.

6. The method of claim 5, wherein the knowledge base is stored using a relational database, and wherein the aggregation of clinical trial statuses and information is cross-referenced according to clinical trial status, disease ontology, gene ontology, chemical structure, biologic, course of treatment, therapy, indication, and treatment effectivity.

7. The method of claim 6, wherein a neural network is employed to cross-reference the aggregation of clinical trial statuses and information.

8. The method of claim 7, wherein the cross-references of the aggregation are used to generate a success score for the identified subset.

9. A computer program product for characterizing the outcome of clinical trials, the computer program product comprising a computer readable storage medium having program instructions embodied therewith, the program instructions executable by a device to cause the device to:
monitor a clinical trial process associated with a set of clinical trial information;
generate, in response to the monitoring, a clinical trial status;
aggregate the clinical trial status and at least a subset of the set of clinical trial information with a plurality of historical clinical trial statuses and information;
generate, from the aggregation, a queryable knowledge base describing each of the aggregation of clinical trial statuses and information; and
notify, in response to the generation of the queryable knowledge base, a user of at least one of the aggregation of clinical trial statuses and information.

10. The computer program product of claim 9, wherein the clinical trial information further includes at least one feature selected from the group consisting of disease ontology, gene ontology, chemical structure, biologic, course of treatment, therapy, and indication.

11. The computer program product of claim 9, wherein the clinical trial status includes an evaluation of the clinical trial process into a completion assessment, wherein the completion assessment is selected from the group consisting of terminated, not pursued, completed, and continued.

12. The computer program product of claim 10, wherein the clinical trial information for each aggregation includes a treatment-indication pair, and wherein notifying the user further comprises:
receiving, from the user, a query including a first treatment-indication pair;
identifying, according to the query, a subset of the aggregation associated with the first treatment-indication pair; and
notifying the user of the identified subset.

13. The computer program product of claim 12, wherein the identifying further comprises generating, from the identified subset, a treatment effectivity for each aggregation within the subset.

14. The computer program product of claim 13, wherein the knowledge base is stored using a relational database, and wherein the aggregation of clinical trial statuses and information is cross-referenced according to clinical trial status, disease ontology, gene ontology, chemical structure, biologic, course of treatment, therapy, indication, and treatment effectivity.

15. A system characterizing the outcome of clinical trials, comprising:
a memory with program instructions included thereon; and
a processor in communication with the memory, wherein the program instructions cause the processor to:
monitor a clinical trial process associated with a set of clinical trial information;
generate, in response to the monitoring, a clinical trial status;
aggregate the clinical trial status and at least a subset of the set of clinical trial information with a plurality of historical clinical trial statuses and information;
generate, from the aggregation, a queryable knowledge base describing each of the aggregation of clinical trial statuses and information; and
notify, in response to the generation of the queryable knowledge base, a user of at least one of the aggregation of clinical trial statuses and information.

16. The system of claim 15, wherein the clinical trial information further includes at least one feature selected from the group consisting of disease ontology, gene ontology, chemical structure, biologic, course of treatment, therapy, and indication.

17. The system of claim 15, wherein the clinical trial status includes an evaluation of the clinical trial process into a completion assessment, wherein the completion assessment is selected from the group consisting of terminated, not pursued, completed, and continued.

18. The system of claim 16, wherein the clinical trial information for each aggregation includes a treatment-indication pair, and wherein notifying the user further comprises:
receiving, from the user, a query including a first treatment-indication pair;
identifying, according to the query, a subset of the aggregation associated with the first treatment-indication pair; and
notifying the user of the identified subset.

19. The system of claim 18, wherein the identifying further comprises generating, from the identified subset, a treatment effectivity for each aggregation within the subset.

20. The system of claim 19, wherein the knowledge base is stored using a relational database, and wherein the aggregation of clinical trial statuses and information is cross-referenced according to clinical trial status, disease ontology, gene ontology, chemical structure, biologic, course of treatment, therapy, indication, and treatment effectivity.
